(19) United States
(12) Patent Application Publication
(10) Pub. No.: US 2005/0260593 A1
(43) Pub. Date: Nov. 24, 2005

(54) Fluorescent labeling reagents with multiple donors and acceptors

(76) Inventors: Shiv Kumar, Belle Mead, NJ (US); Chung-Yuan Chen, Piscataway, NJ (US)

Correspondence Address: AMERSHAM BIOSCIENCES;PATENT DEPARTMENT, 800 CENTENNIAL AVENUE, PISCATAWAY, NJ, 08855, US

(21) Appl. No.: 10/528,862

(22) PCT Filed: Sep. 25, 2003
(86) PCT No.: PCT/US03/30361

§ 371 Date: Mar. 23, 2005

Related U.S. Application Data
(60) Provisional application No. 60/413,517, filed on Sep. 25, 2002.

(51) Int. Cl. C12Q 1/68; C07H 21/04; C07D 311/88; C07D 45/14

(52) U.S. Cl. 435/6; 536/25.32; 548/465; 548/455; 549/226

(57) ABSTRACT

Disclosed is a novel class of fluorescent resonance energy transfer (FRET) labelling reagents, based on and synthesised from easily prepared dye building blocks. The labelling reagents are in the form of “cassettes” which enable their attachment to a wide variety of biological and other materials. A labelling reagent comprises at least two fluorescent dye moieties covalently linked via a linker group and optionally having a target bonding group for attaching the reagent to a target. The energy transfer labelling reagents may be bound to target materials through covalent or non-covalent attachment. The dyes are selected so that the emission spectrum of a first (or donor) dye overlaps the absorption spectrum of a second dye, thereby allowing energy transfer to occur between the dyes. The dye building blocks are 4′, 5′-bis-aminomethyl-fluorescein and/or its 5(6)-carboxylic acid and having the structure (1). In addition to the embodiment of the invention which includes a single donor and a single acceptor fluorochrome, the fluorescent energy transfer labelling reagents according to the invention may further comprise one or more third fluorochromes each having third absorption and emission spectra covalently attached to said first or second fluorochromes.

### CROSS REFERENCE TO RELATED APPLICATIONS

This application claims priority to U.S. provisional application No. 60/413,517, filed Sep. 25, 2002; the disclosures of which are incorporated herein by reference in its entirety.

### BACKGROUND OF THE INVENTION

The present invention relates to fluorescent dyes, more particularly to energy transfer fluorescent dyes with multiple donors and/or acceptors and their applications.

### DESCRIPTION OF RELATED ART

A variety of fluorescent dyes have been developed for labeling and detection of components in biological and other systems. One class of dyes developed and applied extensively to DNA sequencing is fluorescence resonance energy transfer (FRET) based fluorescent dyes, which are constructed of a donor dye and an acceptor dye. Generally, in these dyes, the donor and the acceptor dyes are positioned in close proximity and with proper orientation to each other, the photon energy absorbed by the donor is transferred to the acceptor causing the acceptor fluorophore to fluoresce when excited at the donor absorption wavelength. To ensure the most efficient transfer of energy, it is important that the donor fluorophore has high extinction coefficient, high quantum yield and efficient transfer of the absorbed excitation energy of the donor to the acceptor in the form of acceptor fluorophore emission. Furthermore, for efficient energy transfer, there should be good overlap between the emission of the donor dye and the absorption of the acceptor dye.

A variety of energy transfer fluorescent dyes, mostly involving two fluorophores (as donors and acceptors), have been described in the literature (Proc. Natl. Acad. Sci, USA (1995) 92, 4347-4351, Anal. Biochem. (1995), 231, 131-140, Nucleic Acids Research (1996), 24, 1144-1148, Anal. Biochem. (1996), 243, 15-27, Nucleic Acids Research (1997), 25, 2816-2822, and Tetrahedron Letters (2000) 41, 8867-8871). However, as mentioned previously, the energy transfer is a function of spectral overlap between the emission of the donor and the absorption of the acceptor. When such an overlap is marginal, as shown by fluorescein and a much longer wavelength absorbing fluorescent dye such as Cy5™ (with the emission of fluorescein at 520 nm and the absorption of Cy5 at 650 nm), a low ET results. In such a case, two consecutive energy transfer processes with better spectral overlaps could be more efficient.

This kind of strategy led to recent literature precedence (U.S. Pat. No. 6,008,373 and U.S. Pat. No. 6,130,094) to two consecutive energy transfer process involving fluorescent dyes. Waggoner and co-workers (U.S. Pat. No. 6,008,373) reported an example of two consecutive energy transfers, one from fluorescein to a Cy3 followed by another transfer of energy from the Cy3 to an attached Cy5, which gave a large Stokes' shift of 172 nm. They obtained an even larger Stokes' shift of 282 nm when Cy7 was used as the longest emitting fluorophore. However, no conclusion was made whether two consecutive energy transfer steps were more efficient than a direct one, for instance, from fluorescein to Cy5 or Cy7.

Ju et al (J. Am. Chem. Soc., (2001), 123, 12923-12924 and WO 02/22883) constructed a trichromophore-labeled oligonucleotide that had a scaffold of 26 nucleotides, designated as F-4-R-6-Ct-13. When excited at 488 nm, the predominating emission of the assembly was at 670 nm due to Cy5 and a Stokes' shift of 182 nm with an overall quantum yield of 0.13, while the quantum yield of the Cy5 was 0.27. However, since the extinction coefficient of Cy5 at 488 nm was much less than at peak absorption of 650 nm (less than 2%), this assembly was much brighter than the unmodified dye by a factor of at least 25 when excited at 488 nm.

Despite its usefulness in labeling primers for DNA sequencing and PCR-based genetic analysis, such an assembly cannot be used as a labeling reagent for much more general usage. In this invention, we have designed a labeling reagent with molecular architecture based on 4′,5′-bis-aminomethyl fluorescein as shown in FIG. 1. This molecular design provides close to optimal spacing between donor and acceptor fluorophores and, hence, high energy transfer (ET) efficiency.

The basis of energy transfer is generally accepted as Forster Resonance Energy Transfer (FRET) by a dipole-dipole interaction mechanism proposed by Theodor Forster (Joseph R. Lakowicz, “Principles of Fluorescence Spectroscopy” 2^{nd} Edition, Chapter 13, Kluwer Academic Plenum Publishers, 1999). Forster's theory implies that the closer the donor and the acceptor fluorophores, the better the energy transfer. However, experience has shown that these two fluorophores should not be too close to cause quenching of each other.

Therefore, as in most practical ET applications, linkers were used to keep the fluorophores separated as illustrated in U.S. Pat. No. 5,863,727 and WO 00/13026. This approach was adopted, despite the fact that the introduction of these linkers eventually lengthened the spatial separation between the two fluorophores, and, thus, lowered the efficiency of energy transfer.

In reported literature (Nucleic Acids Research, (1997), 25, 2816-2822), the most optimal separation between donor and the acceptor fluorophores appears to be that shown by the “bifluor-1”, a dye dimer consisting of 5-carboxytetramethyl-rhodamine linked to 4′-aminomethyl fluorescein-5-carboxylic acid. However, “bifluor-1” was not used in DNA sequencing due to considerations such as poor enzyme incorporation and others.

Moreover, the “bifluor-1” structure cannot be used for the transfer of energy between three fluorophores since, after the attachment of two fluorophores onto 4′-aminomethyl fluorescein-5 carboxylic acid, there is no functional group left for the attachment of a biological molecule, such as a nucleotide.

For this reason, we have developed a 4′,5′-bis-aminomethyl-fluorescein as the basic skeleton for our “trifluor-1”. Fluorescent labels based on such a “trifluor-1” structure can be excited optimally at a neon-argon laser at a wavelength of 488 nm and fluoresce at a significantly different wavelength with large Stokes' shifts. Also, with the introduction of a 5- or 6-carboxyl group, the basic structure can be extended in its use for synthesising a fluorescent labeling reagent for various applications.

### SUMMARY OF THE INVENTION

Accordingly, the present invention provides a novel class of fluorescent resonance energy transfer (FRET) labelling reagents, based on and synthesised from easily prepared dye building blocks. The labelling reagents are in the form of “cassettes” which enable their attachment to a wide variety of biological and other materials. A labelling reagent comprises at least two fluorescent dye moieties covalently linked via a linker group and optionally having a target bonding group for attaching the reagent to a target. The target bonding group is chosen to be suitable for forming a covalent linkage with a complementary group on the target material. Alternatively, the energy transfer labelling reagents may be bound to target materials through non-covalent attachment. The dyes are selected so that the emission spectrum of a first (or donor) dye overlaps the absorption spectrum of a second dye, thereby allowing energy transfer to occur between the dyes. The dye building blocks are 4′,5′-bis-aminomethyl-fluorescein and/or its 5(6)-carboxylic acid and having the structure (I).

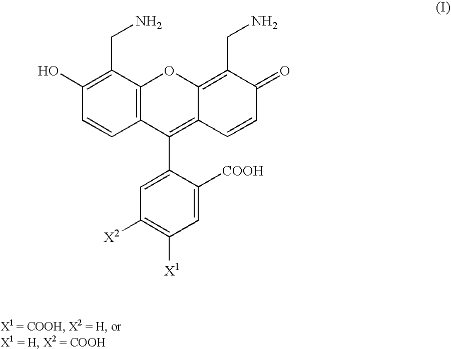

Thus, in a first aspect there is provided a compound comprising:
i) a first fluorochrome having first absorption and emission spectra; and
ii) at least one of a second fluorochrome each said second fluorochrome being covalently attached through a linker group to said first fluorochrome and each second fluorochrome having second absorption and emission spectra, the wavelength of the emission maximum of the second fluorochrome(s) being longer that the emission maximum of the first fluorochrome and a portion of the absorption spectrum of each of said second fluorochromes overlapping a portion of the emission spectrum of said first fluorochrome such that each of said second fluorochromes is capable of accepting energy from said first fluorochrome; and wherein said first fluorochrome comprises a radical of the dye 4′,5′-bis-aminomethylfluorescein having the structure of formula (II):

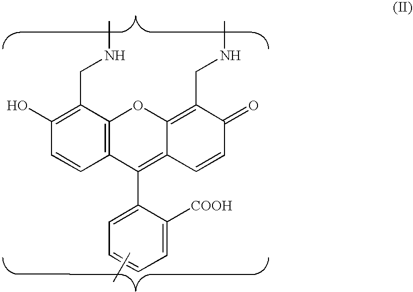

According to the first aspect, the fluorescein chromophore is employed as the donor fluorochrome in a fluorescent energy transfer labeling reagent. To this structure is covalently attached one or more second fluorochromes. The one or more second fluorochromes are in an energy transfer arrangement with the first (or donor) fluorochrome, such that photoexcitation of a first fluorochrome results in the transfer of energy from that dye to the second acceptor fluorochrome(s). Furthermore, additional energy transfers involving one or more additional fluorochrome moieties may also be created. Thus, optionally one or more third fluorochromes may be covalently attached to the “bifluorophore” complex by means of further linker groups. In this arrangement, a portion of the emission spectrum of the second fluorochrome overlaps the absorption spectra of the one or more third fluorochromes. The wavelength of the emission maximum of the third fluorochrome is longer that the wavelength of the second fluorochrome, such that energy absorbed by the first fluorophore(s) upon excitation with light is transferred through the second to the third fluorophore to give an emission wavelength with a very large Stokes' shift.

Preferably, the reagent according to the first aspect includes at least one target bonding group capable of forming a covalent bond with a target material.

The linker group comprises a chain of linked atoms, suitably C₁₋₄₀ alkyl chains, which may optionally include one or more groups selected from —C(O)—, —C(S)—, —NR′—, —O—, —S—, —CR′═CR′— and —CO—NR′— groups, where R′ is hydrogen or C₁₋₄ alkyl. The chain may be optionally substituted, if desired, with groups as known to those skilled in the art which do not prevent energy transfer, for example, C₁₋₄ alkyl, C₁₋₄ alkoxy and halo. The linker group may include part of the constituents extending from the fluorochrome, that is, the linker groups may be derived from functional groups attached to the dye chromophore, suitably the 4′- and/or 5′-aminomethyl groups and/or the 5(6)-carboxylic acid groups attached to the fluorescein chromophore. Thus, while the linker is covalently attached to the dye chromophore, it is not a part of it. Furthermore, none of the linker groups should permit conjugation between donor and acceptor chromophores.

Fluorescent energy transfer labelling complexes according to the present invention show energy transfer ranging from 50% to 99% efficiency. Energy transfer efficiency depends on several factors such as spectral overlap, spatial separation between donor and acceptor, relative orientation of donor and acceptor molecules, quantum yield of the donor and excited state lifetime of the donor. In a preferred embodiment, the fluorochromes may be separated by a distance that provides efficient energy transfer, preferably better than 75%.

In the present invention, the term “radical” is used to define the core structure of the first fluorochrome and is derived from the dye, 4′,5′-bis-aminomethylfluorescein (or its 5(6)-carboxylic acid derivative). Thus, 4′,5′-bis-aminomethylfluorescein forms the molecular building block from which the fluorescent energy transfer reagents are synthesised. Preferred positions for the covalent attachment of further fluorochromes, and optionally other substituents as defined herein are shown in FIG. 1. Furthermore, one or more hydrogen atoms of the aromatic ring structures of the fluorescent dye-radical of formula (II) may be replaced by a substituent group if desired, where the substituent is selected from a halogen (such as fluorine and chlorine), nitrile, hydroxy, thiol, C₁-C₆ alkyl, C₁-C₆ alkoxy and aryl.

The fluorescent energy transfer labelling reagents of the present invention preferably include a target bonding group capable of forming a covalent bond with a target material to enable the reagent to label the material, such as a biological compound. The target bonding group may be linked to the chromophore structure via a linker group, preferably (but not exclusively) derived by chemical modification of the 4′- and/or 5′-aminomethyl groups of 4′,5′-bis-aminomethyl-fluorescein. If 4′,5′-bis-aminomethyl-fluorescein-5(6)-carboxylic acid is used as the dye building block, the 5- or 6-carboxylic moiety may also be chemically modified by well known methods so as to introduce a target bonding group. The target bonding group may be any group suitable for attaching the dye to a target material, such as a carrier material, a biological compound, or a further dye molecule. For example, the target bonding group may be a reactive group that can react under suitable conditions with a complementary functional group of a target material. Alternatively, the target bonding group F may be a functional group and the target may contain the reactive constituent. In either case, the target molecule becomes covalently labelled with the reagent according to the invention. Suitable reactive groups are selected from N-hydroxysuccinimidyl ester, N-hydroxy-sulphosuccinimidyl ester, isothiocyanate, haloacetamide, dichlorotriazine, maleimide, sulphonyl halide, acyl halide, anhydride and phosphoramidite. Suitable functional groups are selected from hydroxy, amino, sulphydryl, and carboxyl groups.

Suitably, the fluorescent energy transfer labelling reagent according to the invention is a compound having the structure (III): 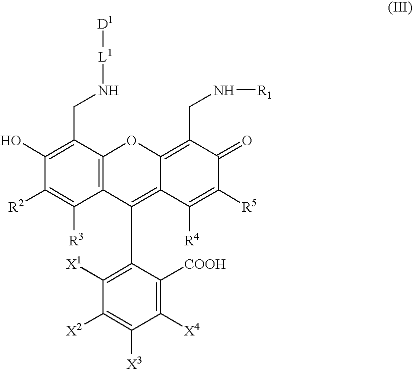
wherein:
D¹ is an acceptor dye selected from the group consisting of xanthine dyes, rhodamine dyes and cyanine dyes;
R¹ is selected from H, an amino-protecting group, the group -L²—F and the group -L²-D², where D² is a fluorescent dye selected from the group consisting of xanthine dyes, rhodamine dyes and cyanine dyes;
R², R³, R⁴ and R⁵ independently represent H, F, Cl, C₁-C₆ alkyl, C₁-C₈ substituted alkyl, C₁-C₆ alkoxy, sulfonate, sulfone, amido, nitrile, aryl or heteroaryl; or R² and R³ and/or R⁴ and R⁵ taken together may be linked to form a fused aromatic or heteroaromatic ring system;
X¹, X², X³ and X⁴ independently represent H, F, Cl, C₁-C₆ alkyl, C₁-C₆alkenyl, C₁-C₆ alkynyl, COOR′, SO₃H, CH₂OH, the group -L³—F and the group -L³-D³, where D³ is a fluorescent dye selected from the group consisting of xanthine dyes, rhodamine dyes and cyanine dyes; and R′ is selected from hydrogen and C₁-C₄ alkyl;
F is a target bonding group; and
L¹, L² and L³ are each a linking group and each independently comprises a group containing from 1 to 40 linked atoms selected from carbon atoms which may optionally include one or more groups selected from —C(O)—, —C(S)—, —NR′—, —O—, —S—, —CR′═CR′— and —CO—NR′— groups, where R′ is hereinbefore defined.

Preferably, the compound of formula (II) includes at least one target bonding group capable of forming a covalent bond with a target material.

Preferably, in the compound of formula (III), each of L¹, L² and L³ independently contains from 1 to 20 atoms.

Preferably, L¹, L² and L³ are each independently:
—{(CHR′)ₚ-Q-(CHR′)ᵣ}ₛ—
where Q is selected from: —CHR′—, —C(O)—, —C(S)—, —NR′—, —O—, —CR′═CR′— and —CO—NR′—; R′ is hydrogen or C₁-C₄ alkyl, each p is independently 0-5, each r is independently 0-5 and s is 1 or 2.

Preferably, Q is selected from —CHR′—, —C(O) and —CO—NH—, where R′, p, r and s are hereinbefore defined.

Specific examples of reactive groups and their complementary functional groups are shown in Table 1.

TABLE 1
Possible Reactive Groups and Functional Groups Reactive Therewith

| Reactive Groups | Functional Groups |
|---|---|
| Succinimidyl esters | primary amino, secondary amino |
| Anhydrides, acid halides | primary amino, secondary amino, |
| | hydroxyl |
| Isothiocyanate | amino groups |
| Vinylsulphone | amino groups |
| Dichiorotriazines | amino groups |
| Haloacetamides, maleimides | thiols, imidazoles, hydroxyl, amines |
| Carboxyl | amino, hydroxyl, thiols |
| Phosphoramidites | hydroxyl groups |

Particularly suitable reactive groups which are useful for labeling target materials with available amino and hydroxyl functional groups include:

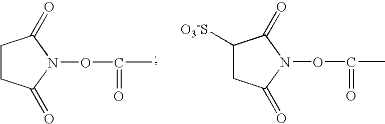

Particularly suitable reactive groups which are useful for labeling target materials with available thiol functional groups include:

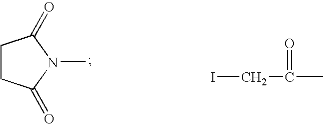

Suitable amino-protecting groups will be well known to the skilled person and include N-alkyl and N-alkenyl derivatives such as N-methyl, N-^{t}butyl and N-allyl; carbamates, such as benzyl carbamate; and N-acyl derivatives, such as N-formyl, N-acetyl and N-benzoyl. Derivatives of the compounds of formula (III) that include amino-group protecting groups will be useful in the synthesis of energy transfer dye labelling reagents based on the molecular building block, 4′,5′-bis-aminomethyl-fluorescein, during attachment of the other fluorophore(s), target bonding groups, solubilizing and charge carrying substituents.

Aryl is an aromatic substituent containing one or two fused aromatic rings containing 6 to 10 carbon atoms, for example phenyl or naphthyl, the aryl being optionally and independently substituted by one or more substituents, for example halogen, straight or branched chain alkyl groups containing 1 to 10 carbon atoms, aralkyl and C₁-C₆ alkoxy, for example, methoxy, ethoxy, propoxy and n-butoxy.

Heteroaryl is a mono- or bicyclic 5 to 10 membered aromatic ring system containing at least one and no more than 3 heteroatoms which may be selected from N, O, and S and is optionally and independently substituted by one or more substituents, for example halogen, straight or branched chain alkyl groups containing 1 to 10 carbon atoms, aralkyl and C₁-C₆ alkoxy, for example, methoxy, ethoxy, propoxy and n-butoxy.

Halogen and halo groups are selected from fluorine, chlorine, bromine and iodine.

Preferred examples of xanthine dyes are selected from fluorescein, naphthofluorescein, rhodol and derivatives thereof.

Preferred examples of rhodamine dyes are selected from 5-carboxyrhodamine (Rhodamine 110-5), 6-carboxyrhodamine (Rhodamine 110-6), 5-carboxyrhodamine-6G (R6G-5 or REG-5), 6-carboxyrhodamine-6G (R6G-6 or REG-6), N,N,N′,N′-tetramethyl-5-carboxyrhodamine, N,N,N′,N′-tetramethyl-6-carboxyrhodamine (TAMRA or TMR), 5-carboxy-X-rhodamine, 6-carboxy-X-rhodamine (ROX).

Preferred examples of cyanine dyes are selected from Cy3 (3-(ε-carboxypentyl)-1′-ethyl-3,3,3′,3′-tetramethyl-5,5′-disulphonato-carbocyanine), Cy3.5 (3-(ε-carboxypentyl)-1′-ethyl-3,3,3′,3′-tetramethyl-4,5,4′,5′-(1,3 disulphonato)dibenzo-carbocyanine), Cy5 (1-(ε-carboxypentyl)-1′-ethyl-3,3,3′,3′-tetramethyl-5,5′-disulphonato-dicarbocyanine, Cy5.5 (1-(ε-carboxypentyl)-1′-ethyl-3,3,3′,3′-tetramethyl-4,5,4′,5′-(1,3-disulphonato)-dibenzo-dicarbocyanine, Cy7 (1-(ε-carboxypentyl)-1′-ethyl-3,3,3′,3′-tetramethyl-5,5′-disulphonato-tricarbocyanine.

In one embodiment of the invention, the fluorescent labelling reagents further comprise one or more water solubilizing substituents attached covalently to the dye chromophore, either directly or indirectly via a suitable linker group. The water solubilising constituents must be unreactive with the target bonding group of the complex. Solubilising groups, for example, sulphonate, sulphonic acid and quaternary ammonium, may be attached directly to aromatic ring structures of the dye chromophores. Alternatively, solubilising groups may be attached by means of a C₁ to C₆ alkyl linker chain to the aromatic ring structures and may be selected from the group —(CH₂)ₖ-W where W is selected from sulphonate, sulphonic acid, quaternary ammonium and carboxyl; and k is an integer from 1 to 6. Water solubility may be advantageous when labelling biological target materials, for example, proteins and nucleic acid derivatives.

Alternatively, a less hydrophilic polar form of the energy transfer reagent may bind non-covalently to DNA by intercalation between the base pairs or by interaction in the minor groove of DNA. Such compounds may be useful for DNA quantitation or localisation. In this embodiment, the fluorescent labelling reagents of the invention further comprise a charge carrying group, suitably a chain containing from 1 to 5 positively charged nitrogen or phosphorus atoms. Some of these positively charged nitrogen or phosphorus atoms may be present in the linker groups, L¹, L² and/or L³. Preferably, the charge carrying group contains positively charged nitrogen atoms, each provided by a quaternary ammonium group, or alternatively a protonated tertiary amino group, a guanidinium group, or a pyridinium group. A particularly preferred charge carrying group is a straight or branched chain containing from 1 to 30 chain carbon atoms said group having the structure:
—(CH₂)ₘN⁺R^{a}R^{a}R^{b}
wherein each R^{a} is independently C₁-C₄ alkyl and R^{b} is C₁-C₄ alkyl or is the group —(CH₂)ₘN⁺R^{a}R^{a}R^{b} where R^{a} and R^{b} are hereinbefore defined and m is an integer from 1 to 4. The additional charge on the labeling complex allows the manipulation of electrophoretic mobility of target molecules labelled with the energy transfer reagents of the invention.

In another embodiment, the fluorescent labelling reagents further comprise two or more first fluorochromes linked in an energy transfer relationship with a second fluorochrome. As shown in FIG. 2, each of the first fluorochromes comprises a radical of the dye 4′,5′-bis-aminomethylfluorescein-5(6)-carboxylic acid and the first fluorochromes are covalently linked head to tail through the 4′- (or 5′-)amino and the carboxyl groups of the radical. The first fluorochrome “complex” contains additional sites that may be utilised for covalent attachment of the second fluorochrome and/or a target material. Upon excitation with light, the fluorescein donor molecules transfer the combined energy absorbed to the second fluorochrome.

In addition to the embodiment of the invention which includes a single donor and a single acceptor fluorochrome, the fluorescent energy transfer labelling reagents according to the invention may further comprise one or more third fluorochromes each having third absorption and emission spectra covalently attached to said first or second fluorochromes. For example, a third fluorochrome may be attached to a second fluorochrome. In this example, the wavelength of the emission maximum of the third fluorochrome is longer than the wavelength of the emission maximum of the second fluorochrome. A portion of the absorption spectrum of the third fluorochrome overlaps a portion of the emission spectrum of the second fluorochrome such that excitation of said first fluorochrome produces fluorescence from the third fluorochrome.

In a still further embodiment of the invention, the fluorescent labelling reagent may contain a plurality of said second fluorochromes, each covalently attached through a linker to said first fluorochrome, each of said second fluorochromes being capable of accepting energy from said first fluorochrome when said first fluorochrome is excited by light. The extinction coefficient of the first fluorochrome is suitably greater than 50,000 Liter/mole cm and the quantum yield greater than 0.5, preferably greater than 0.75. Preferably, the second fluorophore has an extinction coefficient of greater than 40,000 Liters/mole cm and a quantum yield of 0.1 or greater (compared to fluorescein as unity). Furthermore, the third fluorochrome(s), if employed in the labelling complex, should also have an extinction coefficient, preferably greater than 40,000 Liters/mole cm, as well as quantum yield of 0.1 or greater. In one embodiment, energy transfer from donor to acceptor chromophores may be achieved by exciting the fluorophore at 488 nm and then allowing the energy transfer process to generate emission from the longest emitting fluorophore. Alternatively, in a cascade process, both donor and intermediate fluorophores may be excited simultaneously at 488 nm. The energy absorbed by both fluorophores is then transferred to a third fluorophore

In a still further embodiment, the energy transfer reagent may include a chain of fluorescein polymers with acceptor fluorochromes, or other functional groups, attached to different positions on the chain so as to satisfy the requirements for different specific applications as shown in FIG. 3. The acceptors may be the same or may be different as required.

The labeling reagents of the invention are synthesized preferably by covalently linking 4′,5′-bis-aminomethyl-fluorescein-5(6)-carboxylic acid to other fluorophores by known methods to form energy transfer donor-acceptor labelling reagents. The energy transfer reagents may be used to covalently label and thereby impart fluorescent properties to target materials. Thus, in a second aspect, there is provided a method for labelling a target material, the method comprising adding to a liquid containing said target material a fluorescent energy transfer reagent according to the present invention, and incubating said reagent with the target material under conditions suitable for binding to and thereby labelling said target material. The method comprises incubating the target material with an amount of the energy transfer labelling reagent having at least one target bonding group as defined hereinbefore, under conditions to form a covalent linkage between the target and the labelling reagent. Suitable target biological materials include, but are not limited to the group consisting of: antibodies, lipids, proteins, peptides, carbohydrates, nucleotides containing or are derivatized to contain one or more amino, sulphydryl, carbonyl, hydroxyl, carboxyl, phosphate or thiophosphate groups; oxy or deoxy polynucleic acids containing or are derivatized to contain one or more amino, sulphydryl, carbonyl, hydroxyl, carboxyl, phosphate or thiophosphate groups; microbial materials, drugs, hormones, cells, cell membranes and toxins.

In an alternative embodiment, the fluorescent reagents need not have a target bonding group and may be used to bind non-covalently to another compound. For example, the complex may be dissolved, then mixed in an organic solvent with a polymer particle, such as polystyrene then stirred by emulsion polymerization. The solvent is evaporated and the fluorescent dye complex is absorbed into the polystyrene particles.

### BRIEF DESCRIPTION OF THE DRAWINGS

The present invention will be better understood by reference to the drawings in which:

FIG. 1 shows the structure of 4′, 5′ bis-aminomethyl-fluorescein molecular building block and the preferred positions for possible attachment of the other fluorophore(s), target bonding groups, solubilizing and charge carrying substituents, and/or target material.

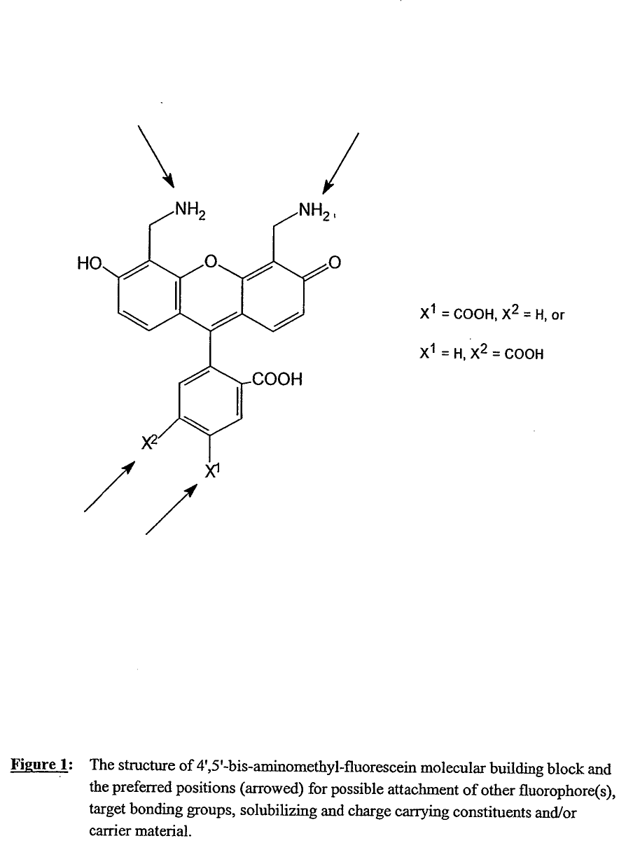

FIG. 2 shows the molecular structure of a dimeric 4′,5′-bis-aminomethyl-fluorescein-5-carboxylic acid and the preferred positions for possible attachment of the other fluorophore(s) and/or other target material.

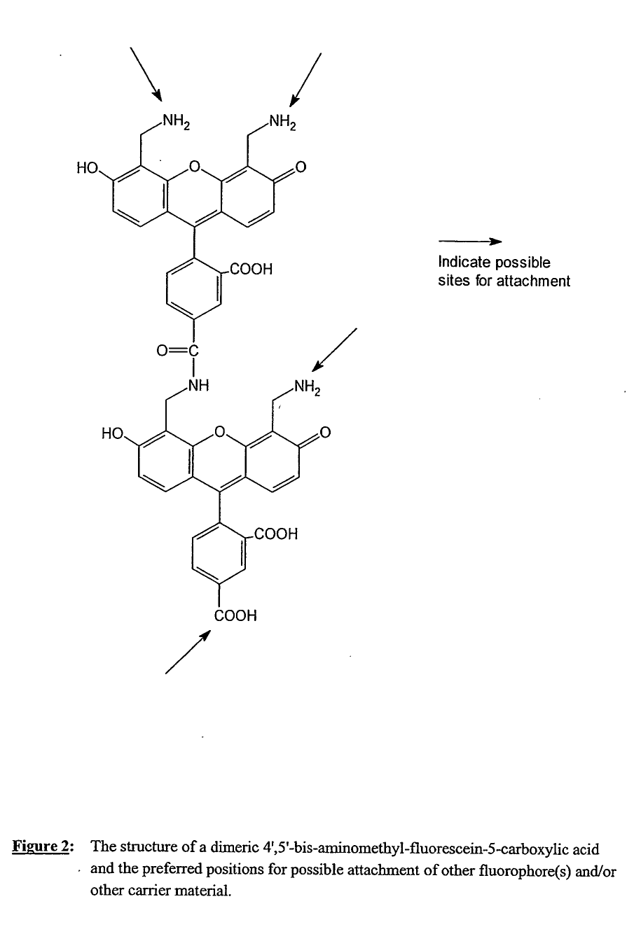

FIG. 3 shows the molecular structure of a polymer of 4′,5′-bis-aminomethyl-fluorescein-5-carboxylic acid and the positions for possible attachment of the other fluorophores(s) and/or other target material.

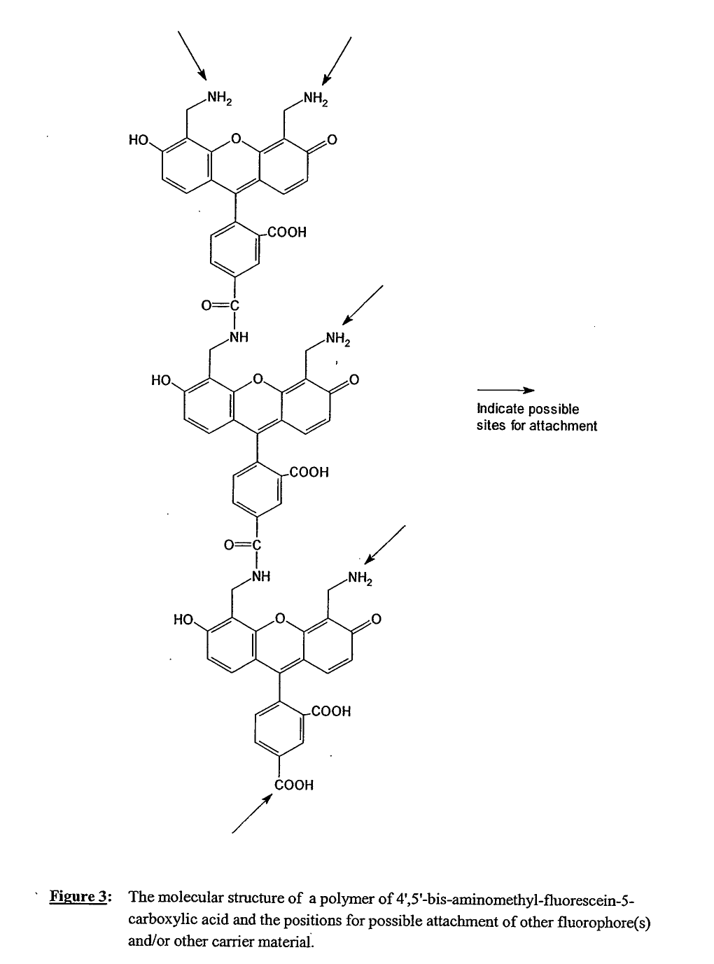

FIG. 4 is a schematic illustration of the overlapping absorption ( - - - ), and emission ( - - - ) spectra of fluorophores suitable for FRET.

FIG. 5 shows the absorption and emission (excitation at 488 nm) spectra of FAM-Cy5 “bifluor” in MeOH/Hunig base.

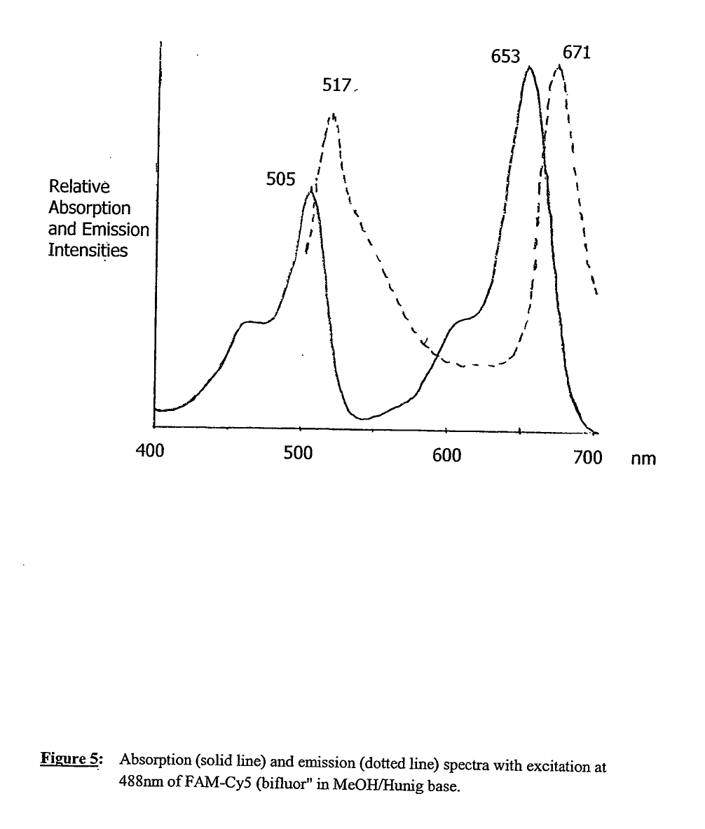

FIG. 6 shows the absorption and emission (excitation at 488 nm) spectra of FAM-TAMRA-Cy5 “trifluor” in MeOH/Hunig base.

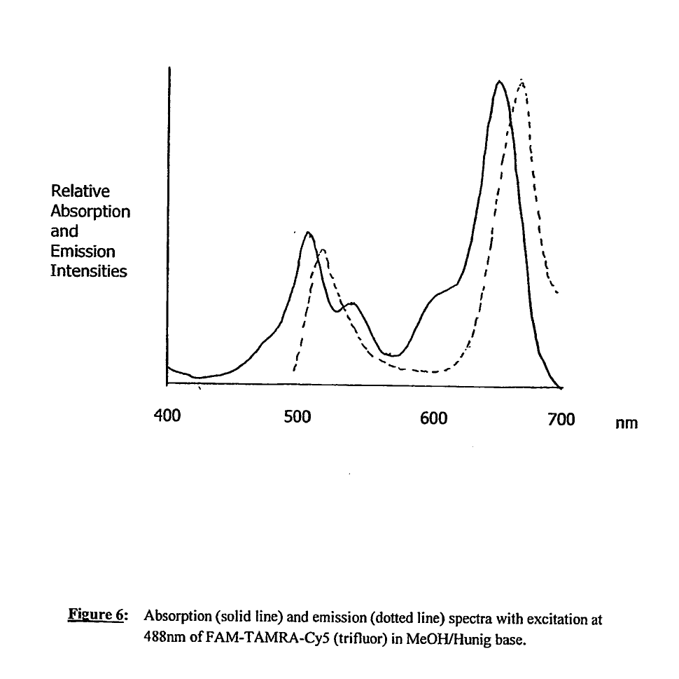

FIG. 7 is a Photon Flow Diagram for Donor-Acceptor Pair (BB). The flow diagram monitors the fate of 100 photons absorbed by the fluorescein donor in the donor-acceptor pair (BB).

FIG. 8 is a Photon Flow Diagram for Trifluor (TA).-Acceptor Pair (BB). The flow diagram monitors the fate of 100 photons absorbed by the fluorescein donor in the donor-acceptor pair (BB).

### DETAILED DESCRIPTION OF THE INVENTION

The present invention provides fluorescent labeling reagents with large Stokes' shifts. For purposes of the present invention, the Stokes' shift of the labelling reagent is the difference in nanometers between the absorption maximum of the shortest wavelength light absorber of the reagent and the emission maximum of the longest wavelength emitter. The energy transfer labelling reagents as hereinbefore described, may contain two or more fluorophores linked together for transfer of energy from a shorter wavelength absorber to a longer wavelength emitter resulting in a large Stokes' shift.

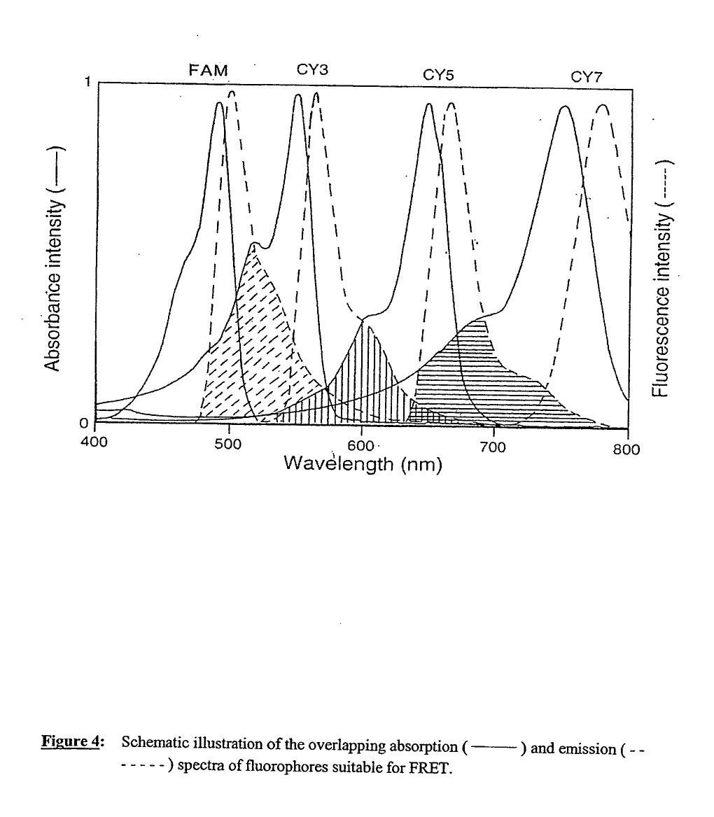

As shown schematically in FIG. 4, the shortest absorbing fluorophore, the first donor fluorophore, absorbs energy upon excitation at an excitation wavelength (solid line) within its absorbance spectrum and emits energy at a wavelength within its emission spectrum (broken line). When linked at an appropriate distance and orientation to a second fluorophore, the first fluorophore transfers, or donates, the energy from its excited state to the second fluorophore at the wavelength within the absorption spectrum (solid line) of the second fluorophore. The second fluorophore accepts the donated energy and emits it at a wavelength within its emission spectrum (broken line), which as shown, is longer in wavelength than the longest wavelength of the emission of the first fluorophore. This process is repeated until the emission for the final, longest wavelength fluorophore ends the chain of energy transfer.

The amount of energy transferred from one fluorophore to the next, does not only depend on the overlap of the emission spectrum of the donor and the absorption spectrum of the acceptor, as illustrated by the shaded area between the first and second fluorophore, shown in FIG. 4. Forester's theory regarding resonance energy transfer predicts that the amount of energy transferred should depend on a spectral overlap term having a fourth power dependency on wavelength of the overlap region. Hence, the energy transfer is more efficient between fluorophores having longer absorption and emission wavelengths.

The fluorescent labeling reagents according to the invention have low molecular weights and can be readily conjugated to antibodies, other proteins, and DNA probes. Low molecular weight as used herein shall mean that the combined molecular weight of the labelling reagent is between 500 and 10,000 Daltons. Therefore, these labeled species will have much greater penetration into intracellular environments than is possible with the larger phycobiliprotein labels currently in use. The low molecular weight fluorescent labeling reagents of the invention should be valuable not only for flow cytometry, but also for laser confocal microscope and for other detection systems requiring multicolor to detection with single wavelength excitation.

The fluorescent labeling reagents preferably include groups capable of forming covalent bonds with corresponding groups on target compounds. Preferably, reactive groups are on the labelling reagent and functional groups are on the target compound or molecule. However, those skilled in the art will is recognize that the functional group may be placed on the labelling reagent and the reactive group may be on the target.

The fluorescent energy transfer dyes according to the present invention may be used in applications that include detecting and distinguishing between various components in a mixture. Thus, the invention also provides a set of two or more different fluorescent energy transfer labelling reagents according to formula (III), wherein each labelling reagent in the set absorbs light energy of substantially the same wavelength and emits (or fluoresces) at a wavelength that is distinguishable from every other reagent in the set. A set of reagents including at least two labelling reagents of the invention may be used in a multiparameter method for detecting target biological compounds present in multiple samples. The method comprises: a) incubating each separate sample with a different label from the set of fluorescent labels to provide fluorescently-labelled samples; b) mixing each of said fluorescently-labelled samples to form a single mixture containing all samples; and c) irradiating the single mixture with a single wavelength excitation source and detecting the fluorescence emissions corresponding to each of the different fluorescently-labelled samples.

Examples of some of the dyes that can be used in the fluorescent labeling reagents of the invention are shown in Table 2. These examples are provided for illustration purposes only and other similar type of dyes may also be used. The following examples should not be construed as limiting the appended claims and the scope of the invention. The current invention should encompass any and all variations that become evident from the teachings provided herein.

TABLE 2

| | Absorption max (nm) | Emission max (nm) |
|---|---|---|
| Fluorophore | (in MeOH) | (in MeOH) |
| Fluorescein | 490 | 520 |
| R110 | 503 | 528 |
| R6G | 520 | 546 |
| TAMRA | 540 | 565 |
| Cy3 ™ | 550 | 570 |
| ROX | 568 | 595 |
| Cy3.5 | 581 | 596 |
| TEXAS RED | 583 | 603 |
| Cy4 | 610 | 628 |
| Cy5 | 649 | 670 |
| Cy5.5 | 675 | 694 |
| Cy7 | 743 | 767 |

### EXAMPLES

1. Synthesis of 4′,5′-bis-Aminomethyl-fluorescein

1.1 4′,5′-bis-(2-Chloroacetamido)-aminomethyl-fluorescein

Fluorescein (3.3 grams) and 2-chloro-n-(hydroxymethyl)-acetamide (5.0 grams) were dissolved in 20 ml of concentrated sulfuric acid. The dark brown solution was stirred at room temperature for two hours. At such time, ESMS⁺ indicated that there was no starting fluorescein left. The product was poured into 200 grams of ice and water and the precipitate was filtered, washed with water, followed by ether and air-dried. NMR of the material, thus obtained, indicated that it was the desired product.

1.2 Hydrolysis of 4′,5′-bis-(2-Chloroacetamido)-aminomethyl-fluorescein

The product from the above reaction was suspended in 40 ml of concentrated hydrochloric acid and heated to reflux for 30 minutes. A clear solution was obtained. The product was evaporated to dryness and the residue recrystalized from methanol/dichloromethane to give the desired product, 4′,5′-bis-aminomethyl-fluorescein, as identified by its NMR and ESMS⁺

2. Synthesis of 4′,5′-bis-Aminomethyl fluorescein-5-carboxylic acid

Since fluorescein-5-carboxylic acid is only sparingly soluble in concentrated sulfuric acid, a modified procedure was employed, as follows.

2.1 4′,5′-bis-(2-Chloroacetamido)-aminomethyl fluorescein-5-carboxylic acid

To 20 ml of concentrated sulfuric acid, stirred at room temperatures, was added dipivaloyl-fluorescein-5-carboxylic acid. To the suspension was added, in portions, excess (4 equivalents) of 2-chloro-n-(hydroxymethyl)-acetamide, until a clear solution was obtained. More of the starting material (both fluorescein and excess 2-chloro-n-(hydroxymethyl)-acetamide) was added until the color of the solution turned from light yellow to brown. The solution was poured onto an ice/water mixture. The precipitate thus obtained, was filtered, washed with water and ether. NMR and ESMS⁺ of the precipitate indicated that it was the desired product.

2.2 Hydrolysis of 4′,5′-bis-(2-Chloroacetamido)-aminomethyl fluorescein-5-carboxylic acid

The product from 2.1 above (1.01 grams) was suspended in 20 ml of concentrated hydrochloric acid and 5 ml of 2-methoxyether. The resulting suspension was heated to reflux and it began to clear in ca. two hours. The solution was, then, allowed to cool. After standing at room temperature overnight, massive precipitation occurred. The precipitate was filtered, washed with 0.1 N hydrochloric acid followed by ether to give the desired product, 4′,5′ bis-aminomethyl fluorescein-5-carboxylic acid, as shown by its NMR and ESMS⁺ spectra.

3. Synthesis of Aminomethyl-FAM-Cy5 bifluor (BB)

4′-Aminomethyl-fluorescein (5 mg) was dissolved in 0.5 ml of dry DMF. To the solution was added 20 mg of Cy5 mono-functional reactive dye in 1.0 ml of sodium bicarbonate-carbonate buffer. After 20 minutes at room temperature, the solvent was evaporated and the residue chromatographed to give the desired product (BB).

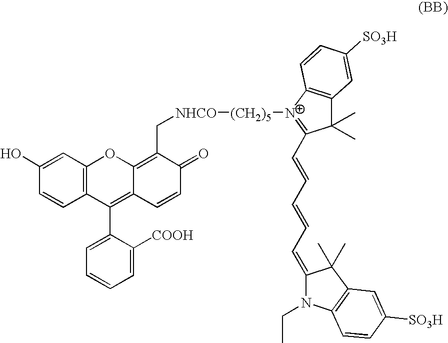

It will be readily appreciated that 4′,5′-bis-aminomethyl fluorescein (prepared by an analogous method as in Example 2) may be used in place of 4′-aminomethyl-fluorescein to prepare 4′,5′-bis-aminomethyl-FAM-Cy5 bifluor, which in turn may be used to prepare an energy transfer labelling reagent having a target bonding group attached at the free 5′-aminomethyl position in the molecule. For example, as shown in reaction Scheme 1, treatment of 4′,5′-bis-aminomethyl-FAM-Cy5 bifluor with succinic anhydride or glutaric anhydride in pyridine affords a carboxylic acid derivatised bifluor dye, which in turn may be converted to its reactive N-hydroxysuccinimidyl ester derivative by reaction with N-hydroxysuccinimide/Dicyclohexyl-carbodiimide in DMF. 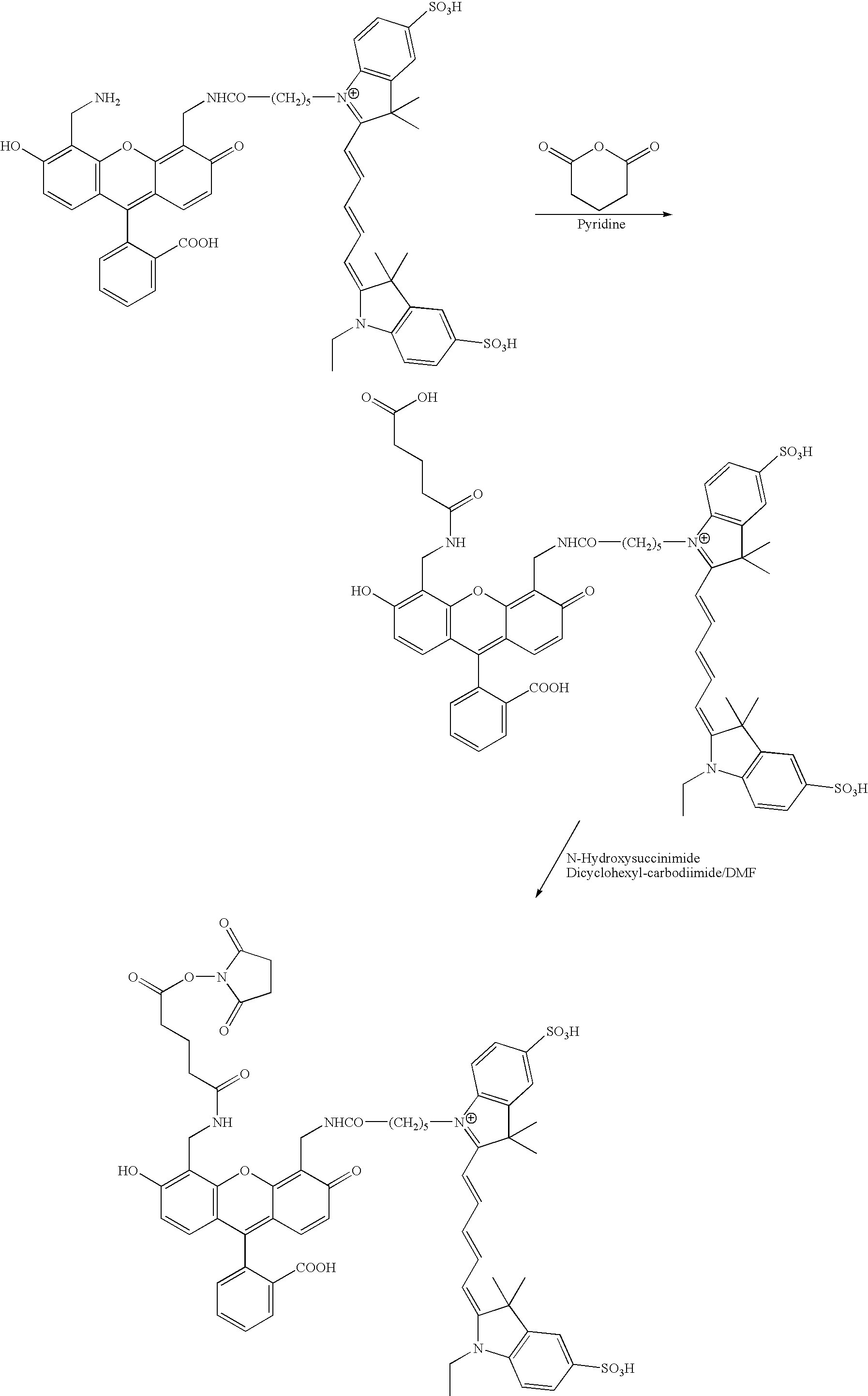
4. Synthesis of Aminomethyl FAM-TAMRA-Cy5 (TA)-“a trifluor”

The synthesis of trifluor, FAM-TAMRA-Cy5 involves the following steps.
4.1. Aminomethyl FAM-TAMRA “bifluor”(BA)

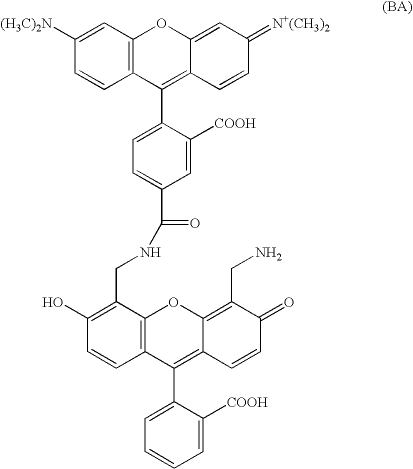

4′,5′-bis-Aminomethyl-fluorescein (4 mg) and 5-carboxytetra-methylrhodamine succinimidyl ester (10 mg) were dissolved in 1 ml of dry dimethylformamide (DMF) with excess N,N-diisopropylethylamine. The reaction was allowed to proceed at room temperature overnight. The product was purified, by TLC, to give the desired the bifluor (BA).
4.2 Aminomethyl-FAM-TAMRA-Cy5 “trifluor” (TA)

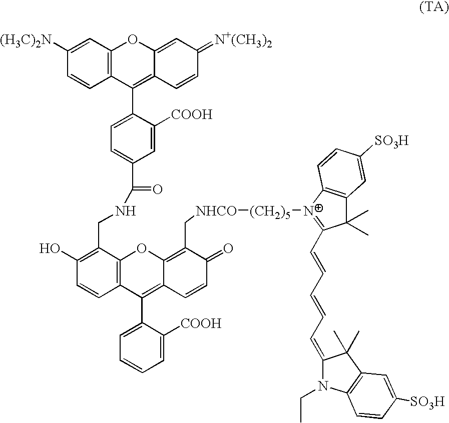

The bifluor (BA), obtained in 4.1 above, was dissolved in dry dimethylformamide with excess N,N-diisopropylethylamine added. To the solution was added a slight excess of Cy-5 mono-functional NHS ester in carbonate/bicarbonate buffer. At the end of the reaction, as shown by the disappearance of the starting material (BA) on thin layer chromatography (TLC), the solvent was evaporated to dryness and the residue chromatographed on a C₁₈ reversed phase TLC plate to give the desired product (TA).

Attachment of Trifluor (TA) to Biological Molecule

4′,5′-bis-aminomethyl fluorescein-5(6)carboxylic acid (prepared as in Example 2) may be used in place of 4′,5′-bis-aminomethyl-fluorescein to prepare 4′,5′-bis-aminomethyl-FAM-5(6)carboxylic acid-Cy5 “bifluor” or Aminomethyl FAM-5(6) acid-TAMRA-Cy5 “trifluor”, which in turn may be used to prepare an energy transfer labelling reagent having a target bonding group attached at the free 5(6)-position of donor fluorescein in the molecule. For example, 4′,5′-bis-aminomethyl-FAM-5(6)carboxylic acid-TAMRA-Cy5 “trifluor” may be converted to its reactive N-hydroxysuccinimidyl ester derivative by reaction with N-hydroxysuccinimide/Dicyclohexyl-carbodiimide in DMF which in turn may be reacted with a target biological molecule. 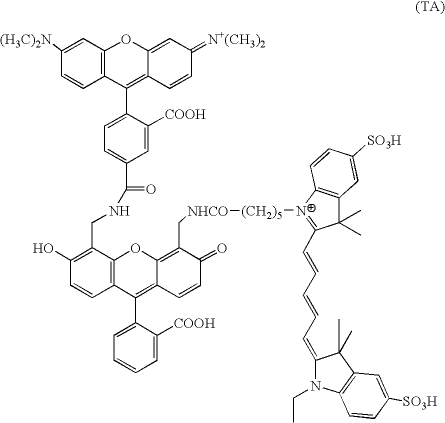
5. Energy Transfer Measurements

5.1 Because of the complexity involved in the practical determination of the extent of energy transfer from a donor fluorochrome to an acceptor fluorochrome, even in a two-fluorophore case, no rigorous, reliable methods for determining energy transfer have been published. In general, according to recent literature, “The efficiency of energy transfer was estimated by calculating the amount of quenching of donor fluorescence that occurs (DQE) when an acceptor is attached”. In another instance, a comparison of the “fluorescence strength” of the donor-acceptor pair was obtained by comparing the intensity of the emission from the acceptor at its emission wavelength, upon excitation at the donor absorption wavelength. Correction for the difference in donor concentration was made by measurement of the concentration of the donor-acceptor pair with absorption at the donor absorption wavelength. This method offers a means to compare the efficiency of energy transfer of donor-acceptor pairs; however, it is limited to the cases where the same donor is involved.

During investigations, it has been found that the first method of estimation, based on DQE, routinely overstates the amount of energy transfer between donor and acceptor, since the amount of loss of energy by the donor is seldom completely transferred to the acceptor. The second direct comparison method does not offer the flexibility of being able to compare donor-acceptor pairs with different donors.

A new method has therefore been developed for determining the portion of the energy absorbed by the donor (and not emitted as donor emission) i.e. that which is transferred to the acceptor. This is the percentage of DQE which is actually emitted by the acceptor. The method involves the measurement of:

i) The absorption and emission characteristics of the donor and the acceptor in the non-conjugated states.

ii) The absorption spectra of the donor-acceptor pair. The optical densities (o.d.) are recorded at the donor absorption wavelength and the acceptor wavelength. For fluorescein-based donor-acceptor pairs, the o.d. at 488 nm is measured, since this wavelength is used to excite the donor-acceptor pairs,

iii) The number of photons emitted by the donor measured at the donor emission wavelength and the number of photons emitted by the acceptor measured at the acceptor emission wavelength upon excitation at the donor absorption wavelength. For fluorescein-based donor-acceptor pairs, the excitation wavelength is 488 nm.

iv) The number of photons emitted by the acceptor in the donor-acceptor pair upon excitation at the acceptor absorption wavelength.

Definitions

i) A slope (SL): is the number of photons emitted, divided by the o.d. of the fluorophore being excited at the excitation wavelength. Since o.d. is directly proportional to number of photons absorbed, the slope is proportional to the quantum yield, which is defined as the number of photons emitted divided by the photons absorbed. Typically, this number is instrument-dependent, depending on its configuration, corrections being required for the excitation source and photomultiplier efficiency. For a particular instrument with preset parameters, the slope measured for a reference compound, such as fluorescein, is close to being a constant within experimental error.

ii) SLDFD is the slope of donor in its free state when excited at the donor absorption wave-length,

iii) SLDCD is the slope of the donor in the donor-acceptor pair when excited is at the donor absorption wavelength,

iv) SLAFA is the slope of the acceptor in its free state, excited at the acceptor absorption wavelength,

v) SLACD is the slope of the acceptor in the donor-acceptor pair when excited at the donor absorption wavelength,

vi) SLACA is the slope of the acceptor in the donor-acceptor pair when excited at the acceptor absorption wavelength,

vii) PQEQ is the percentage of donor quenching, (=DQE¹¹),

viii) PEEA is the percentage quantum yield for the acceptor in the donor-acceptor pair, as compared to that of the free acceptor when excited at the acceptor absorption wavelength, and

ix) PET is the percentage energy transfer of the energy absorbed by the donor to be emitted by the acceptor in the donor acceptor pair when excited at the donor absorption wavelength.

The following calculations may be made.
1. PQEQ (1-SLDCD/SLDFD)×100%,
2. PEEA=(SLACA/SLAFA)×100%
3. PET=(Quantum yield of the donor)×SLACD/SLDFD.
   5.2 Energy Transfer in the Bifluor, Aminomethyl FAM-Cy-5 (BB

As an example, the following values were obtained in the measurement of energy transfer in the donor-acceptor pair, aminomethyl FAM-Cy-5 (BB), (MeOH solvent+one drop of N,N-diisopropylethylamine).
SLDFD=1.41×10⁸
SLAFA=1.39×10⁸
SLDCD=1.12×10⁷
SLACD=1.38×10⁷
SLACA=1.46×10⁸

Thus:
PQEQ=92%
PEEA=106% and
PET=10% (with the quantum yield of fluorescein taken as 1.0).

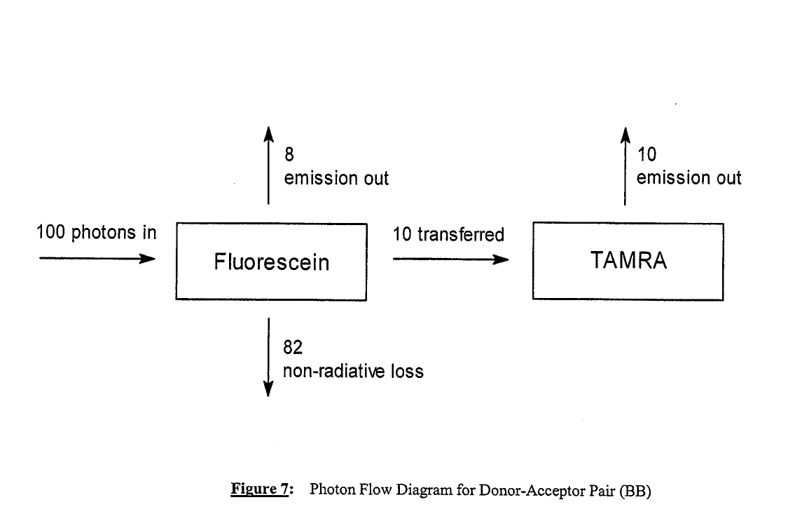

A flow diagram, shown as FIG. 7, may be constructed from the above values, which monitors the fate of 100 photons absorbed by the fluorescein donor in the donor-acceptor pair (BB) and showing the significance of the values. As can be seen from the flow diagram, of the 92 photons loss to the donor upon the absorption of 100 photons, only 10 are transferred to the acceptor. Thus, DQE, as described in U.S. Pat. No. 6,130,094 loc.cit, cannot be used to approximate the energy (photons) transferred to the acceptor.

5.3 Energy Transfer in the Trifluor. Aminomethyl FAM-Cv-5 (BB

The present method for determining energy transfer can be extended to more than two fluorophores in an energy transfer fluorescent labelling reagent. As an example, the following results were obtained for the energy transfer of the trifluor (TA) in MeOH with a trace of N,N-diisopropylethylamine as solvent.
1) PQEQ of the first donor,
2) PEEA of the first acceptor (PEEA₁),
3) PET of the first donor to first acceptor (PET₁),
4) PEEA of the second acceptor (PEEA₂),
5) PET of the first donor to second acceptor (PET₂).
   Thus:
PEQE=90%,
PEEA₁=0%, (No emission from the TAMRA fluorophore in (TA) was observed by excitation either at the fluorescein absorption wavelength or TAMRA absorption wavelength),
PET₁=0%,
PEEA₂=43%,
PET₂=39%

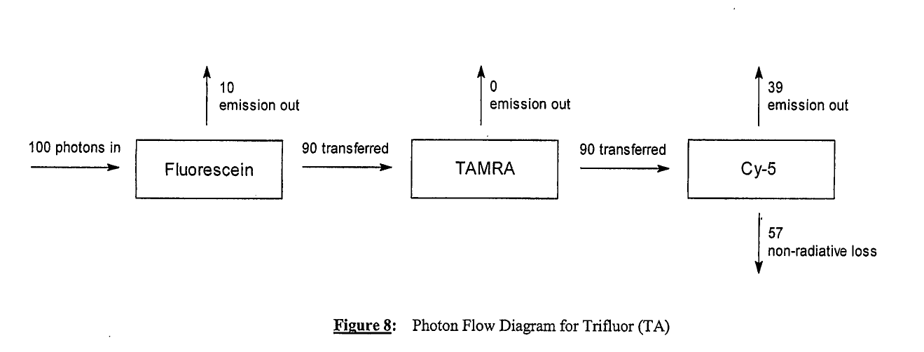

An energy (photon) flow diagram may be constructed, as shown in FIG. 8. By comparing the energy transfer of (BB) and (TA), it can be seen that the introduction of the intermediate fluorophore, TAMRA, improves the energy transfer from fluorescein to Cy5 by a factor of 4. The improvement was obtained as a result of better spectral overlaps in successive energy transfer steps over a single, direct one step energy transfer. Furthermore:

1) The quantum yield of the acceptor/the quantum yield of the donor is equal to the ratio SLAFA/SLDFD. Thus, if the donor is fluorescein, the ratio SLAFA/SLDFD gives the quantum yield of the acceptor (relative to fluorescein as 1.0).

2) PETs as measured are actually the quantum yield of the donor-acceptor pairs excited at the donor absorption wavelength with the emission measured as the acceptor emission maximum. This correlation can be applied to a donor-acceptor pair with multiple acceptors.
6. Synthesis of a Positively Charged Aminomethyl FAM-TAMRA (Donor-Acceptor Pair (BC)

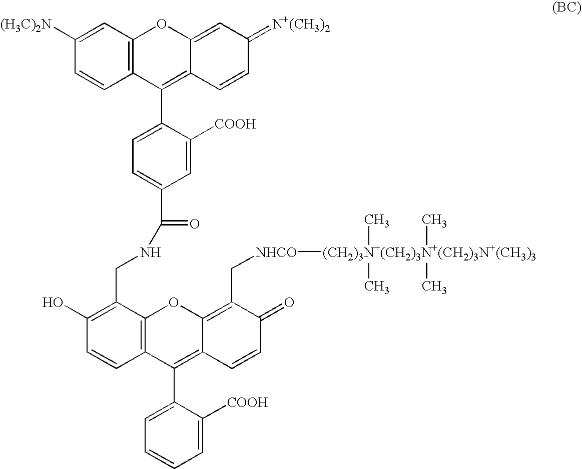

The bifluor (BA) obtained before was dissolved in DMF and reacted with a carbonate/bicarbonate solution of the succinimidyl ester of the acid (CH₃)N⁺(CH2)₃N⁺(CH₃)2(CH₂)₃N⁺(CH₃)₂CH₂COOH at room temperature for 20 minutes. The solvent was removed under vacuum and the residue chromatographed on C₁₈ reversed phase column to give the desired product.

7. Synthesis of an Aminomethyl FAM-TAMRA-terminator (BD)

The preparation of 2′,3′-dideoxycytidine triphosphate labelled with an energy transfer dye, involved the following steps (Reaction Scheme 2).

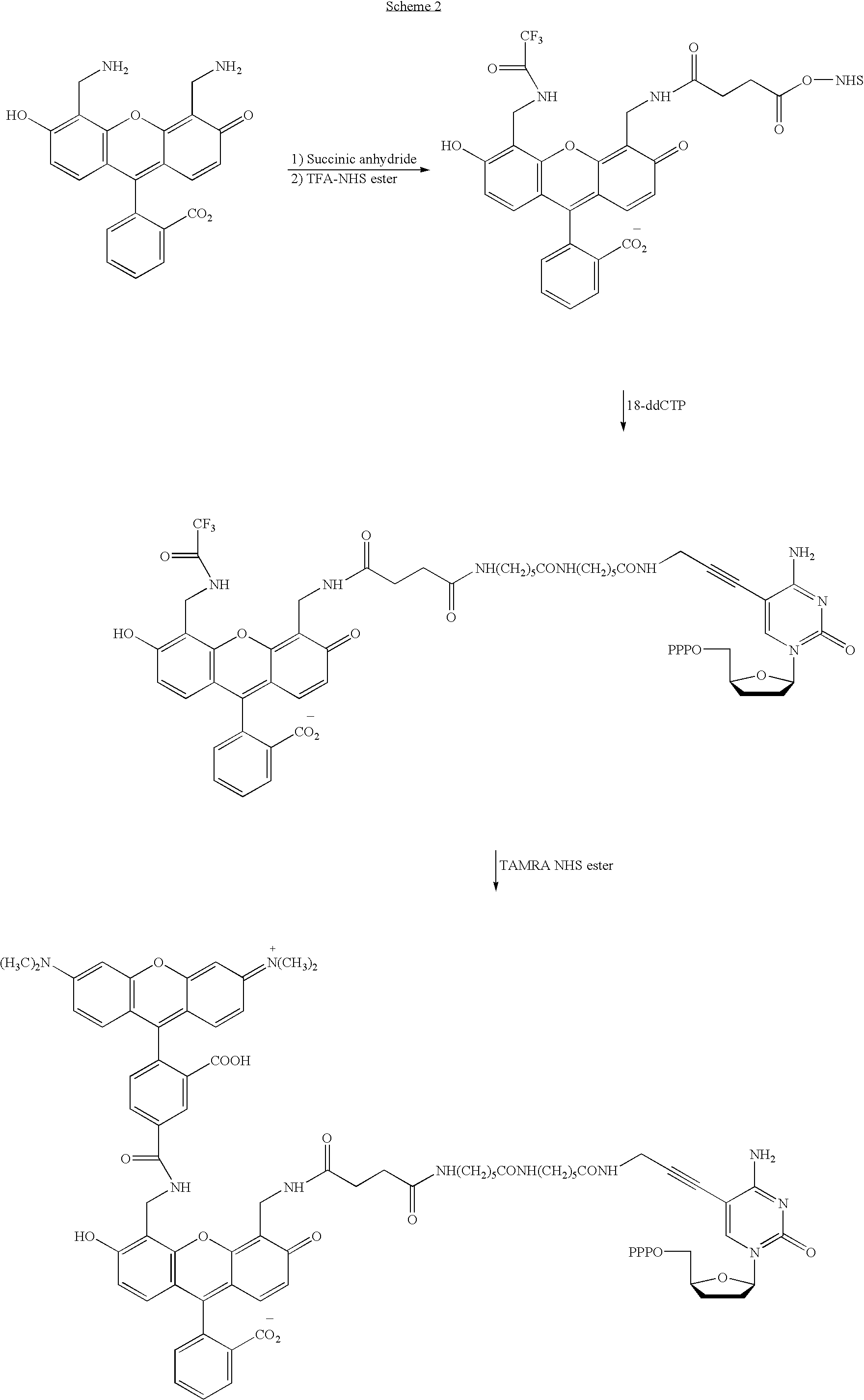

7.1 The 4′,5′-bis-aminomethyl fluorescein obtained as in Example 1 was dissolved in DMF and reacted with succinic anhydride to give the precursor (BE).

7.2 Compound (BE) was reacted with trifluoroacetic acid NHS ester in a mixture of pyridine and dichloromethane to give the intermediate (BG).

7.3 Compound (BG) was dissolved in DMSO and reacted with appropriate linker attached to dideoxycytidine triphosphate in a carbonate/bicarbonate buffer to give a crude product (BH).

7.4 The product obtained in 7.3 was purified and reacted with the TAMRA-NHS ester to give (BF), the final product, which was purified by reverse phase HPLC.

Those skilled in the art having the benefit of the teachings of the present invention as set forth above, can effect numerous modifications thereto. These modifications are to be construed as being encompassed within the scope of the present invention as set forth in the appended claims.

1. A compound comprising:
i) a first fluorochrome having first absorption and emission spectra; and
ii) at least one of a second fluorochrome each said second fluorochrome being covalently attached through a linker group to said first fluorochrome and each second fluorochrome having second absorption and emission spectra, the wavelength of the emission maximum of the second fluorochrome(s) being longer than the emission maximum of the first fluorochrome and a portion of the absorption spectrum of each of said second fluorochromes overlapping a portion of the emission spectrum of said first fluorochrome such that each of said second fluorochromes is capable of accepting energy from said first fluorochrome; and
wherein said first fluorochrome comprises a radical of the dye 4′,5′-bis-aminomethylfluorescein having the formula:

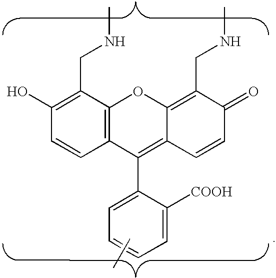

2. The compound according to claim 1 wherein said compound includes at least one target bonding group capable of forming a covalent bond with a target material.

3. The compound according to claim 1 or 2 further comprising:
charge carrying substituents or water solubilizing substituents, covalently attached thereto, or
charge carrying and water solubilizing substituents, covalently attached thereto,
said substituents being unreactive with said target bonding group.

4. The compound according to claim 3 wherein said water solubilizing substituents are selected from the group consisting of amide, sulphonate, sulphate, phosphate, quaternary ammonium, hydroxyl, guanidinium and phosphonate.

5. The compound according to claim 3 wherein said charge carrying substituents incorporate from one to five positively charged nitrogen or phosphorus atoms.

6. The compound according to claim 2 wherein said target bonding group is a reactive group selected from the group consisting of N-hydroxysuccinimidyl ester, N-hydroxy-sulphosuccinimidyl ester, isothiocyanate, haloacetamide, dichlorotriazine, maleimide, sulphonyl halide, acyl halide, anhydride and phosphoramidite.

7. The compound according to claim 2 wherein said target bonding group is a functional group selected from the group consisting of amino, hydroxyl, sulphydryl, and carboxyl groups.

8. The compound according to claim 1 wherein each of said second fluorochromes are selected from xanthine dyes, rhodamine dyes and cyanine dyes.

9. The compound according to claim 2 wherein said target material is selected from the group consisting of: antibodies, lipids, proteins, peptides, carbohydrates nucleotides containing or are derivatized to contain one or more amino, sulphydryl, carbonyl, hydroxyl, carboxyl, phosphate, or thiophosphate groups; oxy or deoxy polynucleic acids containing or are derivatized to contain one or more amino, sulphydryl, carbonyl, hydroxyl, carboxyl, phosphate, or thiophosphate groups; microbial materials, drugs, hormones, cells, cell membranes and toxins.

10. The compound according to claim 1 having a plurality of said second fluorochromes each covalently attached through a linker to said first fluorochrome, and each of said second fluorochromes being capable of accepting energy from said first fluorochrome when said first fluorochrome is excited by light.

11. The compound according to claim 1 further comprising two or more first fluorochromes linked in an energy transfer relationship with a second fluorochrome and wherein each said first fluorochrome comprises a radical of the dye 4′,5′-bis-aminomethylfluorescein-5(6)-carboxylic acid and said two or more first fluorchromes being covalently linked head to tail through the 4′-(or 5′-)amino and carboxyl groups of said radical.

12. The compound according to claim 1 further comprising one or more third fluorochromes covalently attached to said first or second fluorochromes, and each third fluorochrome having third absorption and emission spectra, the wavelength of the emission maximum of said third fluorochrome(s) being longer than the wavelength of the emission maximum of said second fluorochrome and a portion of the absorption spectrum of each of said third fluorochrome(s) overlapping a portion of the emission spectrum of said second fluorochrome such that excitation of said first fluorochrome produces fluorescence from said third fluorochrome(s).

13. A compound having the structure: 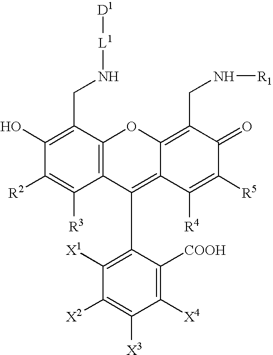
wherein:
D¹ is an acceptor dye selected from the group consisting of xanthine dyes, rhodamine dyes and cyanine dyes;
R¹ is selected from H, an amino-protecting group, the group -L²—F and the group -L²-D², where D² is a dye selected from the group consisting of xanthine dyes, rhodamine dyes and cyanine dyes;
R², R³, R⁴ and R⁵ independently represent H, F, Cl, C₁-C₆ alkyl, C₁-C₆ substituted alkyl, C₁-C₆ alkoxy, sulfonate, sulfone, amido, nitrile, aryl or heteroaryl; or R² and R³ and/or R⁴ and R⁵ taken together may be linked to form a fused aromatic or heteroaromatc ring system;
X¹, X², X³ and X⁴ independently represent H, F, Cl, C₁-C₆ alkyl, C₁-C₆ alkenyl, C₁-C₆ alkynyl, COOR′, SO₃H, CH₂OH, the group -L³—F and the group -L³-D³, where D³ is a dye selected from the group consisting of xanthine dyes, rhodamine dyes and cyanine dyes; and R′ is selected from hydrogen and C₁-C₄ alkyl;
F is a target bonding group; and
L¹, L² and L³ are each a linking group and each independently comprises a group containing from 1 to 40 linked atoms selected from carbon atoms which may optionally include one or more groups selected from —C(O), —C(S)—, —NR′—, —O—, —S—, —CR′═CR′— and —CO—NR′— groups, where R′is hereinbefore defined.

14. The compound according to claim 13 wherein said compound includes at least one target bonding group capable of forming a covalent bond with a target material.

15. The compound according to claim 13 or 14 further comprising:
charge carrying or water solubilizing substituents covalently attached thereto, or
charge carrying and water solubilizing substituents covalently attached thereto,
said substituents being unreactive with said target bonding group.

16. The compound according to claim 15 wherein said water solubilizing substituents are selected from the group consisting of amide, sulphonate, sulphate, phosphate, quaternary ammonium, hydroxyl, guanidinium and phosphonate.

17. The compound according to claim 15 wherein said charge carrying substituents incorporate from two to five positively charged nitrogen atoms.

18. The compound according to claim 13 wherein each of L¹, L² and L³ independently contains from 1 to 20 atoms.

19. The compound according to claim 13 wherein L¹, L² and L³ are each independently:
—{(CHR′)ₚ-Q-(CHR′)ᵣ}s—
where Q is selected from: —CHR′—, —C(O)—, —C(S)—, —NR′—, —O—, —CR′═CR′— and —CO—NR′—; R′ is hydrogen or C₁-C₄ alkyl, each p is independently 0-5, each r is independently 0-5 and s is 1 or 2.

20. The compound according to claim 19 wherein Q is selected from —CHR′—, —C(O)— and —CO—NH—, where R′, p, r and s are hereinbefore defined.

21. The compound according to claim 14 wherein said target bonding group comprises a reactive group for reacting with a functional group on a target material, or a functional group for reacting with a reactive group on a target material.

22. The compound according to claim 21 wherein said reactive group is selected from the group consisting of N-hydroxysuccinimidyl ester, N-hydroxy-sulphosuccinimidyl ester, isothiocyanate, haloacetamide, dichlorotriazine, maleimide, sulphonyl halide, acyl halide, anhydride and phosphoramidite.

23. The compound according to claim 21 wherein said functional group is selected from the group consisting of amino, hydroxyl, sulphydryl, and carboxyl groups.

24. The compound according to claim 13 wherein said xanthine dye is selected from fluorescein, naphthofluorescein, rhodol and derivatives thereof.

25. The compound according to claim 13 wherein said rhodamine dye is selected from 5-carboxyrhodamine (Rhodamine 110-5), 6-carboxyrhodamine (Rhodamine 110-6), 5-carboxyrhodamine-6G (R6G-5 or REG-5), 6-carboxyrhodamine-6G (R6G-6 or REG-6), N,N,N′,N′-tetramethyl-5-carboxyrhodamine, N,N,N′,N′-tetramethyl-6-carboxyrhodamine (TAMRA or TMR), 5-carboxy-X-rhodamine, 6-carboxy-X-rhodamine (ROX).

26. The compound according to claim 13 wherein said cyanine dye is selected from Cy3 (3-(ε-carboxypentyl)-1′-ethyl-3,3,3′,3′-tetramethyl-5,5′-disulphonato-carbocyanine), Cy3.5 (3-(ε-carboxypentyl)-1′-ethyl-3,3,3′,3′tetramethyl-4,5,4′,5′-(1,3-disulphonato)dibenzo-carbocyanine), Cy5 (1-(s-carboxypentyl)-1′-ethyl-3,3,3′,3′-tetramethyl-5,5′-disulphonato-dicarbocyanine, Cy5.5 (1-(ε-carboxypentyl)-1′-ethyl-3,3,3′,3′-tetramethyl-4,5,4′,5′-(1,3-disulphonato)-dibenzo-dicarbocyanine, Cy7 (1-(ε-carboxypentyl)-1′-ethyl-3,3,3′,3′-tetramethyl-5,5′-disulphonato-tricarbocyanine.

27. The compound according to claim 14 wherein said target material is selected from the group consisting of: antibodies, lipids, proteins, peptides, carbohydrates, nucleotides containing or are derivatized to contain one or more amino, sulphydryl, carbonyl, hydroxyl, carboxyl, phosphate or thiophosphate groups; oxy or deoxy polynucleic acids containing or are derivatized to contain one or more of an amino, sulphydryl, carbonyl, hydroxyl, carboxyl, phosphate or thiophosphate groups; microbial materials, drugs, hormones, cells, cell membranes and toxins.

28. A method for labelling a target material comprising:
a) adding to a liquid containing said target material a fluorescent energy transfer reagent according to claim 1 or claim 13; and
b) incubating said reagent with said target material under conditions suitable for binding to and thereby labelling said target material.

29. The method according to claim 28 wherein said target material is selected from the group consisting of: antibodies, lipids, proteins, peptides, carbohydrates, nucleotides containing or are derivatized to contain one or more amino, sulphydryl, carbonyl, hydroxyl, carboxyl, phosphate or thiophosphate groups; oxy or deoxy polynucleic acids containing or are derivatized to contain one or more amino, sulphydryl, carbonyl, hydroxyl carboxyl, phosphate or thiophosphate groups; microbial materials, drugs, hormones, cells, cell membranes and toxins.
